(19) United States
(12) Patent Application Publication
(10) Pub. No.: US 2004/0254092 A1
(43) Pub. Date: Dec. 16, 2004

(54) Surface treatmeat of sars-infected lungs

(76) Inventor: Lin Zhen-Man, Singapore (SG)

Correspondence Address: Lin Zhen Man, Ava Tower 19 07, 10 Ava Road, Singapore, 329949, SG

(21) Appl. No.: 10/469,063

(22) PCT Filed: Jun. 12, 2003
(86) PCT No.: PCT/SG03/00145

(51) Int. Cl. A61K 31/00

(52) U.S. Cl. 514/1

(57) ABSTRACT

SARS infection has wreaks havoc in China, Hong Kong and Taiwan and its effects sending repercussion throughout the entire international society. The death rate has been high and the Chinese and western medical social were quite helpless about this. For more than hundreds of years, lung infectious diseases have always been classified under medical science as internal organs disease. However, today, this paper will point out that this opinion has been misunderstood. There is a need to associate such infection disease with air as an interface. Therefore, SARS infection is a kind surface ulcerous infection.

Since there have been changes from the above-mentioned medical science opinion, we have found the best medical scheme. It will no longer be a dream for SARS infected patients to be discharge from the hospital in a matter of hours. The SARS infected will no longer be life threatening again. Hence, mankind can proudly declare their triumph over SARS.

“Surface Treatment infected of SARS infected lungs” is under the brand new medical concept of the outcome. The “01 Therapy” is the core of the “Surface Treatment”. The sterilizing liquid that is infected into the lung lobes is the surface treatment liquid for 01 therapy of the lungs. The formal name for this liquid is Per Fluoro Chemical (PFC) and the sterilizing is ozone.

### I. BACKGROUND OF THE INVENTION

Since February 2003 years, SARS infection has wreaks havoc in China, Hong Kong and many other countries in the world. Its effects had send repercussion throughout the entire international society. The death rate has been high and the Chinese and western medical social were quite helpless about this. So China. Taiwan, Hong Kong, Singapore and Canada etc. were listed on travel warning district by World Health Organization and pecuniary loss surmount thousand a hundred million, Mankind is faced with death threat.

Knowing how to treat the SARS virus infection had became the top most urgent matter in the Southeast Asia. During this urgent and difficult period of time, the inventors had came up with an innovative medical scheme to save lives, the newest of medical scheme is “Surface Treatment of SARS-Infected Lungs”. Due to the urgency of saving lives, the draft was fax to the Hong kong chief executive and Chinese leader on 15 May 2003. The English version was also forwarded to “WHO-Padey”, “WHO-Liden” by Mey-Verme, Mrs Cnia (WDC) and the leaders who were holding the Geneva meeting on 20 May 2003.

### II. Preface

About the Functions of the Lungs.

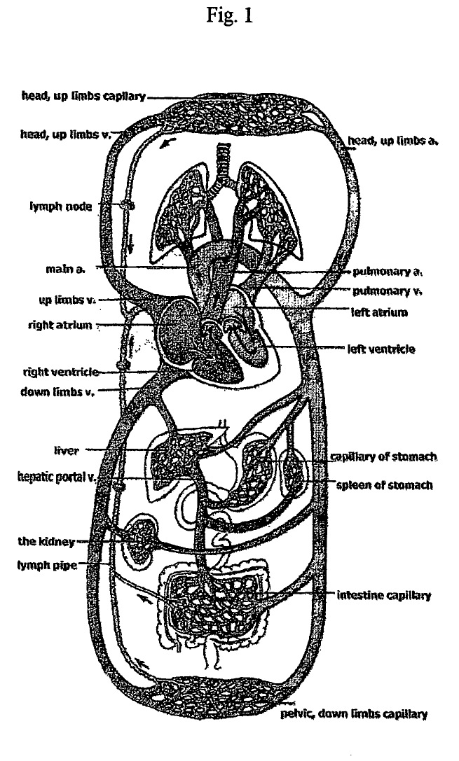

The lungs mainly serve to redistribute the blood from the right ventricle via the lung artery to various lung sub-arteries and capillary vessels in the alveoli, thus achieving gas exchange introducing oxygen and releasing carbon dioxide. Then the blood returns from the lung veins to the left atrium and mixed at a certain proportion in the right ventricle. That is the big circulation of oxygen-containing blood in the arteries providing energy for the body! (FIG. 1.)

Here the medium for gas exchange is not special, just like pumping the air to the bottom of a fish jar to produce bubbles and the oxygen enters the water by rubbing against the external spherical surfaces of the rising bubbles. Our alveoli work like the bubbles in the fish jar and have a large surface area for air contact. The contact area of the dense alveolus tissues in the lungs is up to 70 m ! Tiny blood vessels are spread over the surfaces of these tissues to complete “gas exchange” or, in other words, pulmonary ventilation, via distribution through the blood, interstitial layer and cells. That is the basic idea of the lungs according to modern medicine.

On the medical history, sort of Lung diseases have been numerous. Tuberculosis used to be an infectious disease difficult to cure. However, it can be cured 100% thanks to the discovery of multiple antibiotics. Infant pneumonia is also a common disease, not to speak of pneumococcus. This article describes how to treat SARS.

First, treatment by the traditional Chinese medicine. This method mainly relies on absorption function of the intestines and stomach, which impedes the development of the traditional Chinese medicine. Traditional Chinese prescriptions only help the intestines and stomach to share the burden of the liver, thereby improving only our immunity.

However, the prevailing SARS cures at present are based on Western medicine. The Chinese mainland advocates such antibiotics like tetracycline and erythromycin while Hong Kong regards ribavirin and steroid as effective SARS-containing medicines, but in Canada, which had used Ribavirin for a long time, has now stopped using it because it may have serious side effects.

However, no matter how to, the antibiotics is being absorbed by the intestines and stomach or injected via the veins, they cannot change the subject of the method of transporting anti-bacterium factors in the blood. We call this method blood therapy. Because, many elements in the anti-bacterium factors cannot be absorbed by the intestines and stomach, so the Western medicine takes the lead by this therapy.

That is why the medical circles are focusing on how to improve the efficiency of the “anti-bacterium factors”.

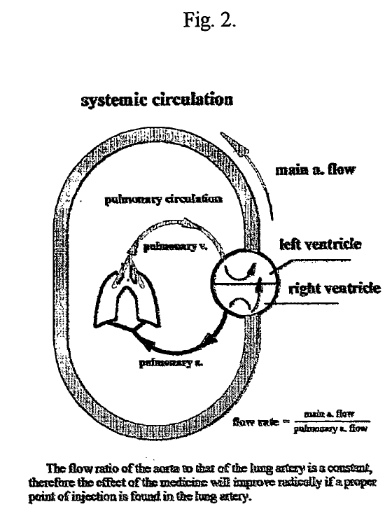

But, as shown in FIG. 2, if the injection point is found in the arteries of the lungs, then the “blood therapy” may become much more effective, as proven by the noticeable flow ratio of the artery and lung circulation. SARS-containing clinical practice is thus more effective. However, we want to point out that the efficiency direction of the “anti-bacterium blood therapy” of SARS is wrong.

As there is a need to define air as an interface, so SARS infection is a kind of surface ulcerous infection. This is a new medical definition, which is likely to revolutionize lung treatment! Therefore we use a familiar industrial term “surface treatment” and to include a technique of supersonic treatment. This is like applying purple liquid medicine to the ulcerous skin which is much more effective than “blood therapy” using any antibiotic.

Up to this point, we can optimistically predict that once the “surface treatment” technique which depends on various antibiotics recommended has found clinic applications, then a SARS patients need only to go to the hospital to have their lungs washed, and SARS will no longer be fatal. At the same time it can also be effective for other pneumonia diseases.

Let's learn something about the physical properties of SARS before dealing with the subject matter of this article—SARS treatment:

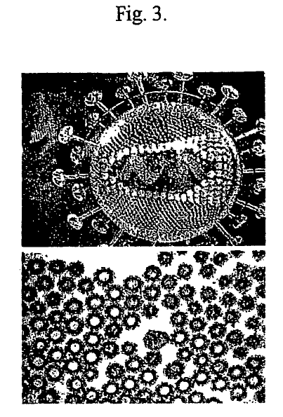

1. FIG. 3 is downloaded from the Internet. SARS virus is smaller than 50 nanometers. SARS virus has numerous crown-like developments, making it absorptive. Overcoming such absorption is significant for the “surface treatment” technique recommended in this article. When we contract bacterium-induced faucitis, we just wet our throat with brine and the pain immediately subsides, because some bacteria are “washed away” by brine, as proven by observing under an electronic endoscope. This traditional inflammation relief method through brine is well-known to all. Inspired by this idea, I think such a simple method can also prevent SARS virus from entering the lungs through the mouth and throat.

2. Super small and super-light virus is visible only through an electronic microscope and the 75-nm N95 standard respirators we use cannot keep out SARS virus, so SARS virus spreads by means of the tiny water droplets and dust particles in the air. In view of that, we can work out a series of effective preventive measures like the “surface treatment” method recommended in this article.

### III. Five Lung “Surface Treatment” Methods

1. Antibiotic gasification and absorption;

2. Massage and sternutation;

3. Taking out and sterilizing lung lobes;

4. Local quick freezing for sterilizing of lung lobes;

5. Injecting sterilizer into lung lobes.

Discussion 1

The method of antibiotic gasification and absorption is not new. This method is effective at the early stage of infection and may serve as a preventive measure before and after medical operation. This method presupposes that the antibiotic in question must be dissolvable in 37° C. water.

Discussion 2

The method of massage and sternutation is more suitably called physical therapy. It works like this: pressing the alveoli by applying force on the lungs and detaching the virus from the cell wall of the alveoli. Facing the nose toward the sun may help to induce sternutation, which is recommendable at the early stage of infection or as a preventive measure. Therefore sunlight sternutation device will be popular on the market. Sternutation is the best exercise for the chest and lungs, and sneezing three times a day is good for senior citizens. The benefits of such an exercise are hardly known but it is a good piece of news for people with weak lungs. This method is just preventive but not effective in detaching the highly absorptive SARS virus.

Discussion 3

Taking out and sterilizing lung lobes is not just a dream. It involves the invention and clinical application of external blood oxygen adding device. This method includes liquid medicine submersion and temperature difference treatment, the latter being the latest medical concept not only suitable for lung patients but also for cancer patients and others. Further exploration of this method may help to replace antibiotic blood therapy with this method:

a. External liquid medicine submersion is more flexible that internal liquid medicine submersion. There are a few or no Liquid medicines that do not damage alveolus tissues. However, an effective liquid medicine for lung lobe submersion will be more effective and attractive if combined with supersonic wave.

b. What is temperature difference treatment? The organs and virus under treatment have difference physiological temperature curves. Temperature difference effect is achieved by selecting a temperature point which is fatal to viruses but from which the organs treated can revive. It is not important whether this method is recorded in medical literature, but the method proves simple, the essential point is the revival rate of the organ under treatment. This is therefore a highly recommended method.

Discussion 4

Local quick freezing and sterilizing of lung lobes is also based on temperature differences but technically it is an improvement from the above three discussions. Taking out lung lobes without cutting off arteries and veins may minimize the damage to the organ and inter-organ contact, making this method practical. While it is difficult to carry out on Lungs, it is feasible for “semi-detached organs” like. The root of the problem is that the quick-freezing equipment involved is not as simple as an ammonia cyclic refrigerator. The clinic freezing device must work in contact mode and is capable of lowering the temperature of an organ of about 1 kg to −30-50° C. within 5˜10 seconds. Many medical fields are gone up and breakthroughs will rely on this kind of technical accomplishment that is made in accordance to the trade circle of science and technology requirement.

Discussion 5

Injecting sterilizer into lung lobes is the subject matter of surface treatment technique of this article. I do not specialize in medicine but just a little medically minded. Inspired by the idea of relieving oral and throat inflammation with brine solution, I managed to find some suitable solvent and sterilizer, but it has to undergo clinical test. But I'm sure that so long as some qualified chemist proposes and there is an adequate range of solvents and sterilizers, SARS will be overcome!

### IIII. O1 Therapy for “Surface Treatment” of the Lungs

The sterilizing liquid injected into lung lobes is the surface treatment liquid for O1 therapy of the lungs. The formal name for this liquid is Per fluoro chemicals (PFC) and the sterilizer is ozone.

This method of introducing supersonic wave with sterilizing liquid may make SARS virus less absorptive and quickly clear viruses in the lungs. This new and practical therapy works like bombing the SARS virus with smart cruise missiles. The missile is single oxygen (O1) separated from ozone, hence “O1 Therapy”!

The effect of the regular antibiotic therapy currently used is limited in that this therapy entails blood exchange, and it is also limited by blood density. For example 50 nm-minus SARS virus is hidden in the middle layer that is inaccessible via the capillary vessels, so the mortality rate of this “blood therapy” is still over 10%. The “blood therapy” of Western medicine has reached its maximum potential. On the contrary, “O1 therapy” is highly effective and is likely to reduce the death rate to zero:

1. Selection of PFE solvent;

2. Properties of ozone sterilizer;

3. Mixing of PFC and ozone;

4. Lung “surface treatment” design flow;

5. Test with animal lung;

6. Special of operating table.

### 1. Selection of PFC Solvent

PFC comes to our mind when we select a liquid medium for cleaning alveoli. Clinical cases are available for PFC breathing technique. We can rely completely on such an effective sterilizer or antibiotic to kill SARS virus. PFC has the characteristics:

1. No color, taste or smell, not poisonous;

2. Low surface tensile strength, not dissolvable in water or fat;

3. High dissolving coefficient for oxygen and carbon dioxide, high density and low solubility, higher dissolving coefficient for ozone;

4. Volatile under indoor temperature and body temperature, does not changeable into other matter via catabolism;

Per fluorine chemical compound (Per fluoro chemicals, PFC) in the innovation medicine application of this case is an important liquid-medium of oxygen for separate out single oxygen-atom. The member-type of PFC are C(5-18)F(12-38), the length of chain of member structure was shown at the number of C, the number of C decides was inner physical special quality, so the Boiling-Point was at 30° C.-215° C. That is to say, the Boiling-Point temperature with the number of C is in proportion. This application seeks to first recommend C6F14 or C7F16, its purpose for volatilizes as soon as possible then pouring into the lung after destroys the germs. But in actual practice of clinical option, more considerations will have to be taken into account, for instance, the level of ulcer by the germs influence and the density of single oxygen-atom and so can; however, then the other fluorine chemical compounds such as C5F9H30 while offering swim-dissociating for the liquid medium of single oxygen-atom, which must emphasize the density effect of single oxygen-atom Depletion Potential.

With the above features, PFC qualifies as a lung surface treatment liquid. It has a dynamic function. On the one hand, oxygen can pass through it to achieve constant gas exchange in the lungs, and on the other hand, the liquid PFC can permeate any alveoli, so that the O1 element in PFC can freely trace SARS virus. The volatility of PFC ensures that no sequela will appear. What is more, PFC can also clean the lungs of damaged cells, cell fragments resulting from inflammation, and SARS virus residuals.

### 2. Characteristics of Ozone Sterilizer

1. The molecule formula of ozone is O3, which is an allotrope of high-energy oxygen and is dissolvable in water and various liquid chemicals;

2. Low-density ozone is colorless and smells like a special grass. It is blue at high temperature and its density is 1.5 times that of air;

3. Ozone sterilizes by releasing single oxygen atom to oxidize and damage the cell of the virus, leaving pure O2, which is beneficial to the lungs;

4. Ozone dissolved in water sterilizes more forcibly and quickly, and it is dissolvable in liquid PFC;

5. When the density of ozone exceeds a certain limit, its sterilizing function is just a matter of seconds;

Therefore, ozone is a good choice as an alveoli sterilizer. The following figures are cited from world-recognized experiment documentation for ozone sterilizing.

| | | | | | Types of | |
|---|---|---|---|---|---|---|
| | | | | | viruses and | Sterilizing |
| | Density | | Time | | pathogens | efficiency |
| Ozone | 10 | mg/m3 | 20 | mins | Type-B | 99.99% |
| sterilizing | | | | | hepatitis | |
| | | | | | surface antigen | |
| | | | | | (HbsAg) | |
| | 0.5 | ppm | 5 | mins | Type-A flu | 99% |
| | | | | | virus | |
| | 0.13 | mg/L | 30 | seconds | Poliomyelitis | 100% |
| | | | | | virus type I | |
| | | | | | (PVI) | |
| | 40 | μg/L | 20 | seconds | Coliphage ms2 | 98% |
| | 0.25 | mg/L | 1 | minute | SA-H and | 99.60% |
| | | | | | human-wheel | |
| | | | | | virus type 2 | |
| | * 12.6 | mg/L | 4 | minutes | Coronaviridae | 100% |
| | 4 | mg/L | 3 | minutes | HIV | 100% |
| | 8 | mg/m3 | 10 | minutes | Mycoplasma, | 99.85% |
| | | | | | Chlamydia, and | |
| | | | | | other pathogens | |

* Red indicates every liter of lung surface treatment solution contains 12.6 mg ozone, which may serves as a reference when we consider the test dosage of ozone.

### 3. Mixing of PFC and Ozone

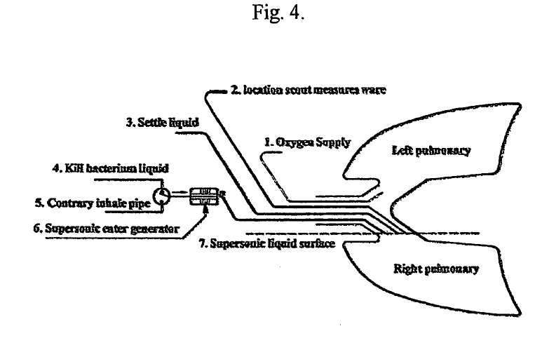

The working process-method of mixing the PFC and ozone are shown in FIG. 4. FIG. 4-1 shows the ozone supply; FIG. 4-2 shows the O3 Contriver; FIG. 4-3 shows the PFC supply; FIG. 44 shows the passageway valve of liquid; FIG. 4-5 shows the pump of gas and liquid mixing; FIG. 4-6 is the mix vessel; FIG. 4-7 is the digital type tester of ozone density which has the export-ability to brake of the working of the mixing pump; FIG. 4-8 is the controller of altitude of liquid and have the export-ability to the working of the passageway valve of liquid.

### 4. Lung “Surface Treatment” Flow

The treatment flow takes the treatment for example of the right lung, while reserving the breath of the left lung for the time being. The final purpose is to treat both lungs at the same time. Process 3 can only be used only after process 4. Test it with animal lung, before applying it on human. It must be noted that the test with animal lung is intended to prove that it applies to process 3, the human body treatment. The advantage of the reverse sequence is time saving.

a. Surface treatment clinic (must be professional anesthetist except for bio-chemical test of body energy): (FIG. 5)

b. Surface treatment clinic scheme diagram: (FIG. 6)

### 5. Test With Animal Lung

Test with animal lung includes two stages: test with one lung of the baby pig and test with both lungs. This process simulates process 3, as specified below:

a. Inject pure PFC into three without virus influence of baby pig:

| | Pure PFC injection 10 mins | | | Pure PFC injection 30 mins | | | Pure PFC injection 120 mins | | |
|---|---|---|---|---|---|---|---|---|---|
| | Change of | | | Change of | | | Change of | | |
| | blood | | | blood | | | blood | | |
| Baby | oxygen | Heart | Symptom | oxygen | Heart | Symptom | oxygen | Heart | Symptom |
| pig | amount | pulses | description | amount | pulses | description | amount | pulses | description |
| 1 | | | | | | | | | |
| 2 | | | | | | | | | |

b. Inject 12.6 mg/L PFC into three virus-free pig to test its reaction to high-density ozone:

| | Pure PFC injection 10 mins | | | Pure PFC injection 30 mins | | | Pure PFC injection 120 mins | | |
|---|---|---|---|---|---|---|---|---|---|
| | Change | | | Change of | | | Change of | | |
| | of blood | | | blood | | | blood | | |
| Baby | oxygen | Heart | Symptom | oxygen | Heart | Symptom | oxygen | Heart | Symptom |
| pig | amount | pulses | description | amount | pulses | description | amount | pulses | description |
| 1 | | | | | | | | | |
| 2 | | | | | | | | | |
| 3 | | | | | | | | | |

c. Inject 25.2 mg/L PFC into three virus-free pig to test its reaction to high-density ozone:

| | Pure PFC injection 10 mins | | | Pure PFC injection 30 mins | | | Pure PFC injection 120 mins | | |
|---|---|---|---|---|---|---|---|---|---|
| | Change | | | Change of | | | Change of | | |
| | of blood | | | blood | | | blood | | |
| Baby | oxygen | Heart | Symptom | oxygen | Heart | Symptom | oxygen | Heart | Symptom |
| pig | amount | pulses | description | amount | pulses | description | amount | pulses | description |
| 1 | | | | | | | | | |
| 2 | | | | | | | | | |
| 3 | | | | | | | | | |

d. Inject 12.6 mg/L PFC into three infected of baby pig to test its reaction to high-density ozone:

| | Pure PFC injection 10 mins | | | Pure PFC injection 30 mins | | | Pure PFC injection 120 mins | | |
|---|---|---|---|---|---|---|---|---|---|
| | Change of | | | Change of | | | Change of | | |
| | blood | | | blood | | | blood | | |
| Baby | oxygen | Heart | Symptom | oxygen | Heart | Symptom | oxygen | Heart | Symptom |
| pig | amount | pulses | description | amount | pulses | description | amount | pulses | description |
| 1 | | | | | | | | | |
| 2 | | | | | | | | | |
| 3 | | | | | | | | | |
| Note 1. | | | | | | | | | |
| The above a-c tests are intended to test whether PEC solvent with or without ozone has bad effect on the lungs. In test c, the density of ozone can be further increased until a reliable pig lung reaction curve, which may serve as a reference for chemists for preparing prescriptions for human treatment. | | | | | | | | | |
| Note 2. | | | | | | | | | |
| Test d is intended for SARS inflammation, needing an infected pig. Tests with difference densities can be worked out by analogy, but the baby pig under the test is much more resistant to diseases than man. Usually, after 1-3 medicine reaction tests, similar results can be obtained in the tests with various dosages and can be observed under a microscope, and the bio-chemical lab can work out a guided report for the chemists in a short time. | | | | | | | | | |
| # The test planning is for your reference only. | | | | | | | | | |

### 6. Important Points in Designing the Operation Table

The operation table should be designed such that it can turn horizontally so that the patient on the table can turn left or right with an angle of at least 45 degrees to facilitate the treatment of the left and right lungs.

### V. CONCLUSION

From the viewpoints mentioned above, the ability of ozone to kill virus was recognized worldwide, ozone application in the PFC solvent was confirmed by 1 of remark of the famous production businesses, the PFC solvent used in the lung had also supported by 2-8 of remark of medical document. Therefore, combining the PFC solution and ozone together will attack the SARS virus in no time. The [method] medicine scheme will definitely treat the SARS virus infection and there will be no side effect at all. This invention will save many lives and change medical-history for lung Infection-Disease.

The handling effect of difference in temperature of invention's theory can also kill dead cancer-cell under completely no side effect, the effect of medical treatment cannot compared with Electrotherapy and Chemotherapy, hence the invention bring Gospel for cancer patient, it was so far the brand-new medical concept and brand-new medical method of “Frozen-Therapy”

### -end-

Remark of Paper/WebPages:

1. http://cms.3m.com/cms/US/en/2-68/iFcFiFM/view.jhtml

2. http://www.vghtpe.gov.tw/˜clinmed/ (Taiwan 89 [symbol] ) [Chinese]

Paper in international journals:

3. Jeng M J, Kou Y R*, Sheu C C, Hwang B. Effects of Exogenous Surfactant Supplementation and Partial Liquid Ventilation on Acute Lung Injury Induced by Wood Smoke Inhalation in Newborn Piglets. Crit Care Med 2003; 31:1166-1174

4. Jeng M J*, Yang S S, Wolfson M R, Shaffer T H. Perfluorochemical (PFC) Combinations for Acute Lung Injury: An in Vitro and in Vivo Study in Juvenile Rabbits. Pediatr Res 2003;53:81-88.

5. Jeng M J*, Oliver R, Wolfson M R, Shaffer T H. Partial liquid ventilation: effect of initial dose and redosing strategy in acute lung injury. Pediatr Crit Care Med 2002;3:163-171.

6. Jeng M J*, Kou Y R, Sheu C C, Hwang B. Effects of partial liquid ventilation with FC-77 on acute lung injury in newborn piglets. Pediatr Pulmonol 2002; 33:12-21.

7. Jeng M J*, Trevisanuto D, Weis C M, Fox W W, Wolfson M R, Shaffer T H. The role of ventilation strategy on Perfluorochemical (PFC) evaporation from the lungs. J Appl Physiol 2001; 90: 1365-1372.

8. Trevisanuto D, Jeng M J*, Weis C M, Fox W W, Wolfson M R, Shaffer T H. Positive end-expiratory pressure modulates perfluorochemical evaporation from the lungs. Biol Neonate 2003;84:53-58.

A. (Use for medicine patent by patent-law of country only)
1. The liquid medicine name of “Surface Treatment of SARS-Infected Lungs” is Per Fluoro Chemicals (PFC) mixing ozone forming a medicine.
2. In the claim 1, the liquid includes all liquids of fluorine element.
3. In the claim 1, includes any substitute liquid to mixing ozone or the single oxygen is decompose by other element.
4. In the claim 1 of liquid medicines include the option of mixing any chemical that might kill or restrain the germs, for instance any antibiotics or other bactericide and so on.
5. In the claim 1, includes any other lung diseases and SARS inflammation.

B. (Use for medicine and methods of medical treatment patent by patent-law of country, for instance US patent office and so on.)
1. The main characteristic of the “Surface Treatment of SARS-Infected Lungs” is to inject sterilizing liquid into the lung lobes.
2. “Surface Treatment of SARS-Infected Lungs” in claim 1, the formal name for the medicine of sterilizing liquid is Fluoro Chemicals (PFC) adding ozone forming a medicine.
3. “Surface Treatment of SARS-Infected Lungs” in claim 1, including any other lung diseases and SARS inflammation.
4. “Surface Treatment of SARS-Infected Lungs” in claim 1, to add antibiotics or other bactericide into the sterilizing liquid to suppress or to kill the virus.
5. In the claim 1, the liquid includes all liquids of fluorine element.
6. In the claim 1, includes any substitute liquid to mixing ozone or the single oxygen is decompose by other element.
7. The claims include a brand-new medical theories that is “The handling effect of difference in temperature” for cure cancer.
8. The claims include a brand-new methods of medical treatment of “Frozen-Therapy” for kill dead the cancer-cell.
